(19) United States
(12) Patent Application Publication
(10) Pub. No.: US 2011/0054502 A1
(43) Pub. Date: Mar. 3, 2011

(54) METHOD FOR CLOSING AN APERTURE IN A PATIENT'S BODY

(75) Inventors: Tatyana Ventura, Palo Alto, CA (US); William James Harrison, Signal Mountain, TN (US)

(73) Assignee: MD-ART, INC., Palo Alto, CA (US)

(21) Appl. No.: 12/549,109

(22) Filed: Aug. 27, 2009

(51) Int. Cl. A61B 17/12

(52) U.S. Cl. 606/158

(57) ABSTRACT

The invention generally relates to a system for closing an aperture in a patient, such as an aperture in a vessel wall of a patient. In certain embodiments, the invention provides a system for closing an aperture in a patient including a delivery device that is attachable to and removable from an exterior of an introducer sheath; and a clip releasably disposed within the delivery device.

### FIELD OF THE INVENTION

The invention generally relates to a system for closing an aperture in a patient, such as an aperture in a vessel wall of a patient.

### BACKGROUND

Catheterization and interventional procedures, such as angioplasty and stenting, generally are performed by inserting a hollow needle through a patient's skin and muscle tissue into the vascular system. A guide wire then is passed through the needle lumen into the patient's blood vessel. The needle is removed and an introducer sheath is advanced over the guide wire into the vessel. A catheter typically is passed through the lumen of the introducer sheath and advanced over the guide wire into position for a medical procedure. The introducer sheath therefore facilitates insertion of various devices into the vessel while minimizing trauma to the vessel wall and minimizing blood loss during a procedure.

Upon completion of the medical procedure, the catheter and introducer sheath are removed, leaving an aperture in the vessel. Commonly, external pressure is applied until clotting and wound sealing occurs. However, this procedure is time consuming and expensive, requiring as much as an hour of time from a physician or nurse, is uncomfortable for the patient, and requires that the patient be immobilized in the operating room, catheterization laboratory, or holding area. Furthermore, a risk of hematoma exists from bleeding prior to hemostasis.

Various apparatuses have been developed for sealing a vascular aperture by occluding, clipping, or suturing the aperture of the vessel. A problem with these prior art devices and techniques is that the introducer sheath must be removed prior to using the closure apparatus to close the aperture. By introducing a new device through the existing puncture site, there becomes an increased risk of contaminating the vessel with skin flora, thereby increasing the chance of infection. Further, the requirement of removing the introducer sheath and then providing the closure apparatus prolongs the intervention.

There is an unmet need for systems and methods that provide for vascular aperture closure that do not require introduction of additional apparatuses or the removal of the introducer sheath at the end of a surgical intervention to achieve closure of an aperture in a patient.

### SUMMARY

The invention generally relates to universal closure systems, devices, clips, and methods that allow for closure of an aperture in a patient, for example, arterial wound closure after femoral artery catheterization. Systems and methods of the invention reduce time for hemostasis and time of patient immobility in the cardiology catheter room, angiography suite, or operating room, thereby reducing hospital stay, and a patient's personal discomfort. The delivery device and/or clip is compatible with standard medical devices, such as introducer sheaths and guiding catheters, is easy to use, and allows the operator to attach the delivery device and/clip on any sheath or guiding catheter being used prior to beginning or at the end of the procedure and close the aperture in the vessel upon removal of the sheath or guiding catheter from a patient. Features of the invention (bioabsorbable clip, compact delivery system, universal compatibility, low cost, easy use) address previous problems in the vascular closure field.

Systems of the invention for closing an aperture in a patient generally include a delivery device that is attachable to and removable from an exterior of an introducer sheath, and a clip releasably disposed within the delivery device. Systems of the invention may further include an introducer sheath. The introducer sheath may be the existing introducer sheath already implanted in a patient to perform a surgical intervention. The delivery device can be attached to the introducer sheath prior to beginning a surgical intervention. Alternatively, the delivery device can be attached to the introducer sheath after starting a surgical intervention, without removal of the sheath from the patient. The delivery device is generally clipped to the exterior of the sheath, although other attachment methods can be envisioned by one of skill in the art.

The delivery device is generally situated at a proximal portion of the sheath prior to and during a surgical intervention. Upon completion of the surgical intervention, the delivery device is advanced to a distal portion of the sheath for deployment of the clip. The clip can be deployed without removal of the sheath from the patient.

The clip can be any type of clip that is suitable to be deployed within the body of a patient and close an aperture in the patient. Exemplary clips include vascular clips and surgical clips. In a preferred embodiment, the clip is a vascular clip.

The clip can include a resilient body having a first ring portion, a second ring portion, and at least one mid-region joining the first and second portions, the body having a compressed delivery configuration and an expanded deployed configuration, and at least one tissue engaging member disposed about each of the first portion and the second portion of the body. The clip is expandable from a delivery configuration in which the clip is loaded within the delivery device to a deployed configuration in which opposite ends of the clip are directed inward towards each other. The clip can be bioresorbable or bioabsorbable. Tissue engaging members of the clip can further include barbs.

In the delivery configuration, the clip is configured to exert a positive pressure on walls of a delivery device, thereby maintaining the clip within the delivery device until deployed. In the deployed configuration, the clip is configured to engage tissue and close an aperture in a patient's body, such as an aperture in a vessel wall. In the deployed configuration, the body of the clip substantially defines a plane and tissue engaging members on the first and second portions are directed inward toward each other. In the deployed configuration, the tissue engaging members on the first and second portions of the body of the clip lie beneath the plane defined by the body. In the deployed configuration, the tissue engaging members of the first and second portions can interlock with each other. The first and second portions of the body of the clip can include a different number of tissue engaging members.

In certain embodiments, the body of the clip is a unitary body. In other embodiments, the mid-region of the clip is spring loaded. Each of the first ring portion and second ring portion can have any shape. Exemplary shapes include a circle, a polygon (regular or irregular), or a modified polygon.

The delivery device can further include a mechanical force regulator. The regulator generates an audible and tactile click during deployment of the clip. The delivery device can further include a pusher sleeve and a constraining sleeve. Each of the pusher sleeve and the constraining sleeve include a body and a handle. The handle of each of the pusher sleeve and the constraining sleeve can be flexible. In certain embodiments, at least a portion of the body of the pusher sleeve is configured to slidably fit within the body of the constraining sleeve. In other embodiments, the pusher sleeve is slidably disposed within the constraining sleeve and the pusher sleeve is flush against the constraining sleeve.

The delivery device can further include a stopper that extends around at least a portion of the device, in which the stopper is positioned at a distal end of the device to prevent the pusher sleeve from advancing into a vessel. The delivery device can further include a protective sheath disposed along an interior of the delivery device, in which the protective sheath is capable of being peeled away from the delivery device after the delivery device has been attached to the introducer sheath or other medical device.

The clip can be deployed by pushing the pusher sleeve, while holding stationary the constraining sleeve, to advance the clip from the delivery device. The delivery device can be configured such that a distal end of the device is tapered such that tissue engaging members of the clip simultaneously contact an exterior wall of a vessel upon deployment of the clip. The clip, in the delivery configuration, can be configured such that upon deployment of the clip from a delivery device, the tissue engaging members of the clip simultaneously contact an exterior wall of a vessel. In certain embodiments, the clip closes the aperture in the vessel by attaching to an exterior wall of the vessel.

In certain embodiments, the delivery device is shaped as a tubular channel having a lateral opening disposed along its length. In other embodiments, the delivery device has a C-shaped cross section. In certain embodiments, the clip has a semicircular shape when it is disposed within the delivery device. In other embodiments, the clip has a C-shape when it is disposed within the delivery device. In other embodiments, the body of the clip has a C-shaped cross section and tissue engaging members on the first and second portions are directed away from each other and toward the tissue to be engaged. These configurations allow the delivery device to be attached to and removed from the introducer sheath or any other medical device.

Another aspect of the invention provides a method for closing an aperture in a vessel in a patient's body, the method including advancing a first medical device through an aperture in a vessel, advancing a delivery device distally along an exterior surface of the medical device to contact an exterior of a vessel wall; and deploying a clip that engages tissues portions adjacent to the aperture in the vessel and closes the aperture in the vessel upon withdrawal of the first medical device from the aperture.

The method can further include, prior to advancing the first medical device through the aperture in the vessel, attaching the delivery device to an exterior surface of the first medical device. The method can further include, after advancing the first medical device through the aperture in the vessel, attaching the delivery device to an exterior surface of the first medical device.

The method can further include introducing at least a second medical device through the introducer sheath into the vessel. The second medical device can be any medical device needed to perform the desired surgical intervention. Exemplary second medical devices include an angioplasty balloon, an atherectomy device, an IVC filter, an angiography catheter, or a stent delivery device. The method can further include performing a surgical intervention within the patient's body using the second medical device introduced through the introducer sheath into the vessel. The method can further include delivering a tissue sealant into the aperture.

Another aspect of the invention provides a method for closing an aperture in a vessel in a patient's body including advancing a medical device through an aperture in a vessel, attaching a delivery device to an exterior surface of the medical device after the medical device has been advanced through the aperture in the vessel, advancing the delivery device distally along the exterior surface of the medical device to contact an exterior of a vessel wall, and deploying a clip from the delivery device that engages tissues portions adjacent to the aperture in the vessel and closes the aperture in the vessel upon withdrawal of the medical device from the aperture.

### BRIEF DESCRIPTION OF THE DRAWINGS

FIG. 1 is a drawing showing an embodiment of a clip and closure system of the invention.

FIG. 2 is a drawing showing an embodiment in which the delivery device is attached to an introducer sheath prior to beginning a surgical intervention.

FIG. 3 is a drawing showing an embodiment in which the delivery device is attached to an introducer sheath after a physician starts a surgical intervention.

FIG. 4 is a drawing showing a side view of an embodiment of a delivery device of the invention.

FIG. 5 is a drawing showing a top view of the delivery device shown in FIG. 4.

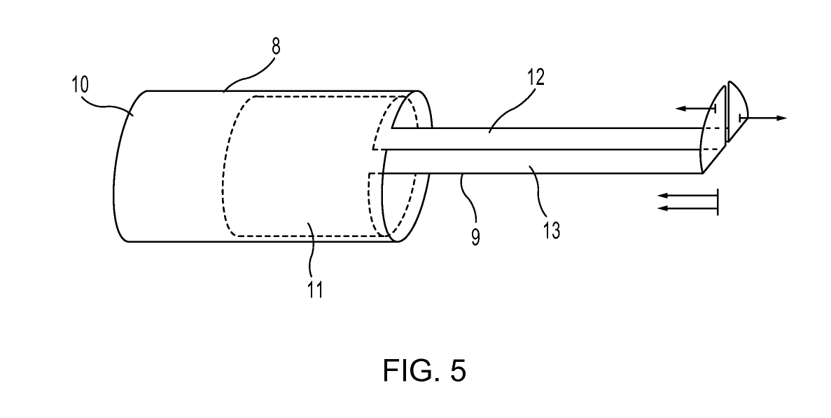

FIG. 6 panel A is a drawing showing an embodiment of a delivery device having a straight distal end. FIG. 6 panel B is a drawing showing an embodiment of a delivery device having an angled distal end.

FIG. 7 panel A is a drawing showing a clip loaded into a delivery device having a straight distal end. FIG. 7 panel B is a drawing showing a clip loaded into a delivery device having an angled distal end.

FIG. 8 is a drawing showing a delivery device with a protective sheath spanning only a portion of the length of the interior of the delivery device.

FIG. 9 is a drawing showing a delivery device with a protective sheath spanning a full length of the interior of the delivery device.

FIG. 10 is a set of drawings showing an embodiment of a clip of the invention. Panel A shows the clip in a deployed configuration. Panels B and C show the clip in a delivery configuration.

FIG. 11 is a drawing showing exemplary shapes and exemplary sizes of tissue engaging members.

FIG. 12 is a drawing showing a magnified section of the clip of FIG. 10. This drawing shows interlocking of tissue engaging members on the left and right portions of the clip in the deployed configuration.

FIG. 13 is a drawing showing an embodiment of the clip in which the clip is compressed into a delivery configuration and loaded within a delivery device.

FIG. 14 is a set of drawings showing different views of an embodiment of a clip of the invention in a delivery configuration. Panel A is a ⅓ lateral view, panel B is a lateral view, and panel C is a front view.

FIG. 15 is a set of drawings showing the body of a delivery device of the invention having grooved channels.

FIG. 16 is a set of drawings showing an embodiment of a clip of the invention from different views and in different configurations. Panel A is a lateral view of the clip in a delivery configuration. Panel B is a lateral view of the clip in a deployed configuration. Panel C is a front view of the clip in the deployed configuration.

FIG. 17 is a set of drawings showing another embodiment of a clip of the invention from different views and in different configurations. Panel A is a lateral view of the clip in a delivery configuration. Panel B is a front view of the clip in the delivery configuration. Panel C is a front view of the clip in a deployed configuration.

FIG. 18 is a drawing showing a magnified view of a tissue engaging member on a clip of the invention.

FIG. 19 is a drawing showing a tab that holds together the handles of the pusher sleeve and the constraining sleeve of the delivery device to prevent premature and/or inadvertent deployment of the clip.

FIG. 20 panels A and B are a set of drawings showing a stopper positioned at a distal end of the constraining sleeve, preventing the pusher sleeve from advancing into the vessel.

FIG. 21 shows a drawing of a delivery device including a mechanical force regulator.

FIG. 22 shows a drawing of a delivery device including a displacement limiter.

A fuller understanding of the aspects, objects, features, and advantages of certain embodiments according to the invention will be obtained and understood from the following description when read together with the accompanying drawings, which primarily illustrate the principles of the invention and embodiments thereof. The drawings are not necessarily to scale and measurements provided in the drawings are exemplary and are not intended to limit the invention in any regard. Like reference characters denote corresponding or related parts throughout the several views. The drawings and the disclosed embodiments of the invention are exemplary only and not limiting on the invention.

### DETAILED DESCRIPTION

Systems and devices constructed in accordance with the present invention provide vascular introduction and wound closure in a single device, eliminating the time and manipulation required to insert a separate closure device at the completion of a procedure.

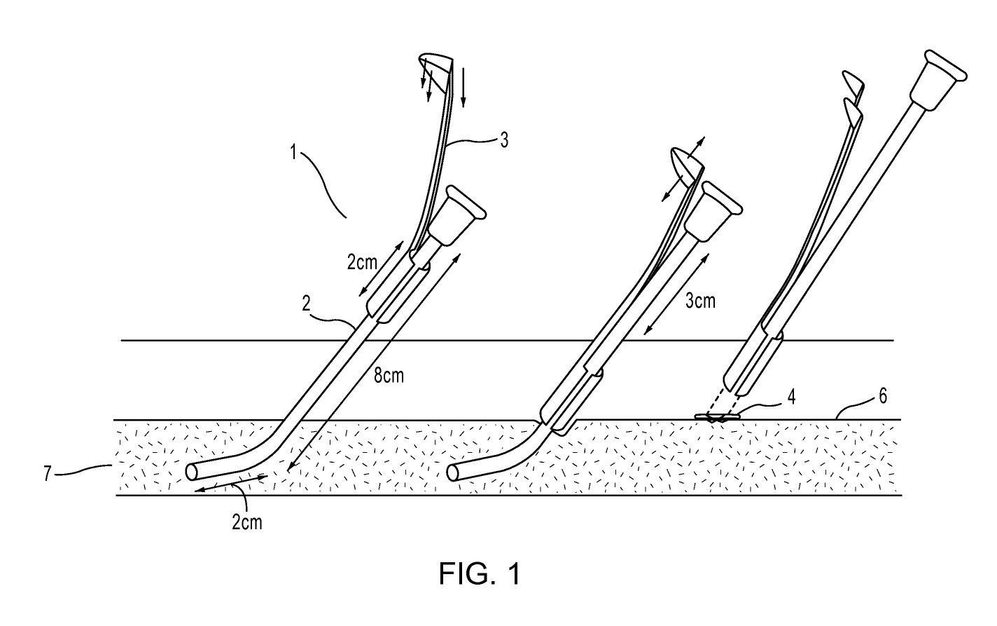

Referring to FIG. 1, a system of the invention 1, includes a delivery device 3, and a clip 4 disposed in the delivery device 3. Systems of the invention can further include an introducer sheath 2. While FIG. 1 shows an introducer sheath 2, the delivery device 3 can be clipped to any medical device, such as a catheter. Introducer sheath 2 is made from a material typically used for vascular introducer sheaths, such as polyethylene or nylon, and includes central lumen 5 through which other devices may be introduced in the vasculature, for example, to perform a diagnostic or interventional procedure such as angiography, angioplasty or stenting.

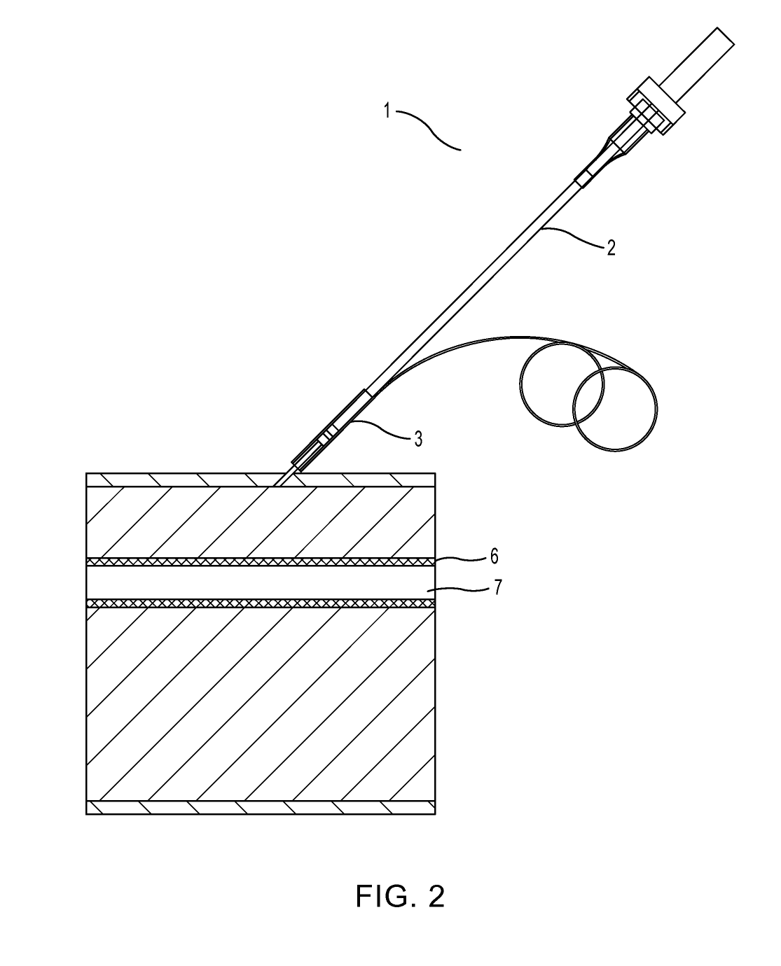

Delivery device 3 is attachable to and removable from introducer sheath 2. The delivery device can be attached to the introducer sheath prior to beginning a surgical intervention. FIG. 2 shows attachment of the delivery device 3 to the introducer sheath 2 prior to the beginning of the surgical intervention. In this embodiment, a physician assembles the preloaded clip device and delivery system to the exterior of the introducer sheath, over a distal end of the sheath prior to introduction to the patient. Once loaded onto the introducer sheath and pulled to the proximal end, the introducer can be used as normal through the entire intervention, without regard to the closure clip system. The clip can be delivered to the vessel via the delivery system, as the physician is completing the procedure. In this embodiment the physician pre-plans the use of the device.

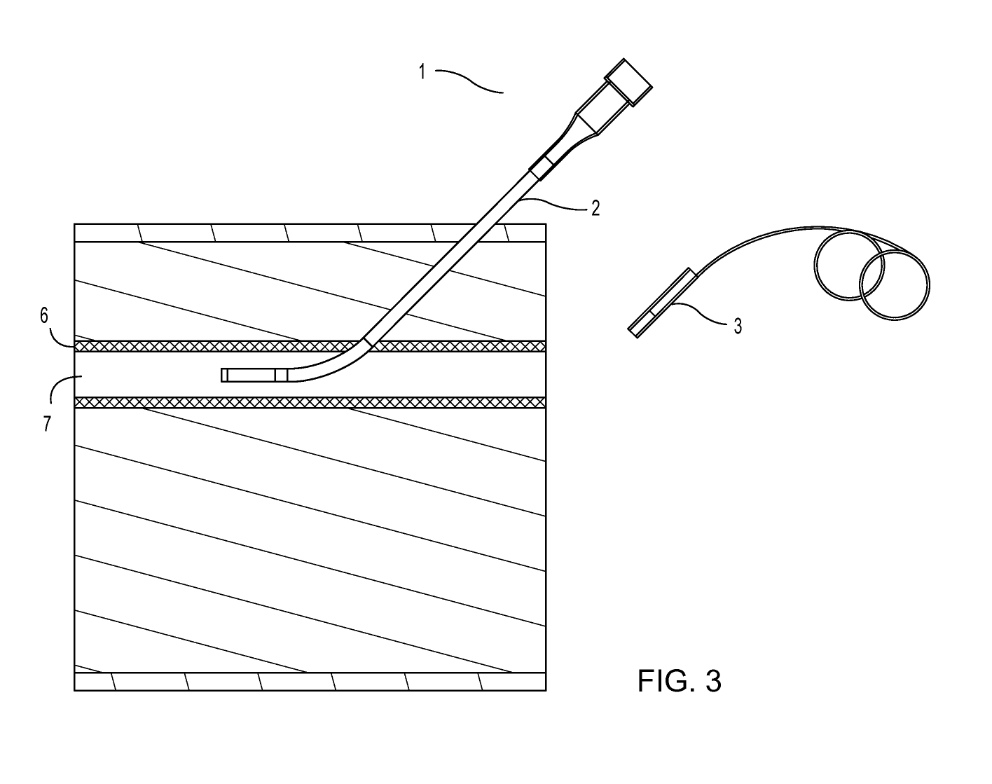

Alternatively, the delivery device can be attached to the introducer sheath after a physician has started a surgical intervention. FIG. 3 shows attachment of the delivery device 3 to the introducer sheath 2 after a physician has started a surgical intervention. In this embodiment, the physician can place the delivery device 3 onto the introducer sheath 2 after the interventional procedure has begun, without removal of the introducer sheath 2 from a patient's body. This system can be clipped onto the sheath, to then be delivered to the vessel via the delivery system, as the physician is completing the procedure.

As mentioned above, the delivery device 3 is pulled to a proximal end of the introducer sheath 2 so that the introducer can be used as normal through the entire intervention (FIG. 1, panel A). Upon completion of the surgical procedure, the delivery device 3 can be advanced along the exterior of the introducer sheath 2 until contact with the vessel wall 6 occurs for deployment of the clip 4 (FIG. 1 panels B and C). As shown in FIG. 1 panel C, the clip 4 is deployed prior to removal of the introducer sheath 2 from a patient's body. Upon deployment of the clip 4, the clip attaches to an exterior of a wall 6 of a vessel 7. Upon removal of the system and the introducer sheath the aperture in the vessel wall 6 is closed.

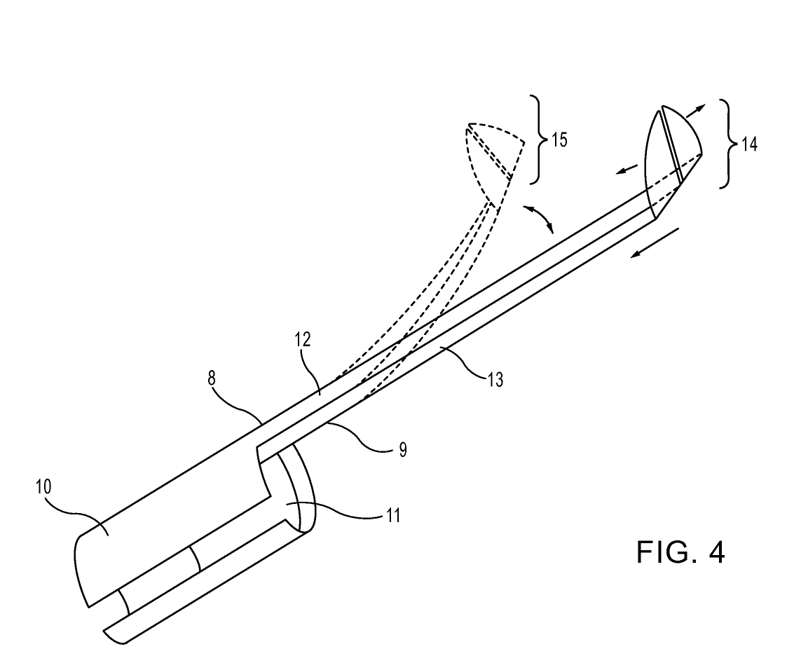

FIGS. 4 (side view) and 5 (top view) show an embodiment of the delivery device 3 of the invention. The delivery device 3 includes a constraining sleeve 8 and a pusher sleeve 9. Each of the constraining sleeve 8 and pusher sleeve 9 include a body (10 and 11) and a handle (12 and 13). The delivery device is configured such that at least a portion of the body 11 of the pusher sleeve 9 is configured to slidably fit within the body 10 of the constraining sleeve 8. In certain embodiments, the pusher sleeve 9 is flush against the constraining sleeve 8. The handles (12 and 13) of the constraining sleeve and the pusher sleeve are flexible (as exemplified in FIG. 4 by the handles in a first position 14 and the handles in a second position 15) and thus allows for the delivery device to be moved so that a physician can use the introducer through the entire intervention, without regard to the delivery device 3.

The body 10 of the constraining sleeve 8 and the body 11 of the pusher sleeve 9 are each formed as a tubular channel having a lateral opening disposed along its length. This can appear as a C-shaped cross-section of the delivery device 3. Because of the shape of the delivery device 3, the delivery device 3 can be attached to introducer sheath 2, for example, an exterior surface of the sheath, prior to starting or after beginning a surgical intervention. Additionally, the shape allows for the delivery device 3 to be attached to or removed from any medical device, i.e., a universal delivery device that is suitable to mate with any standard medical device.

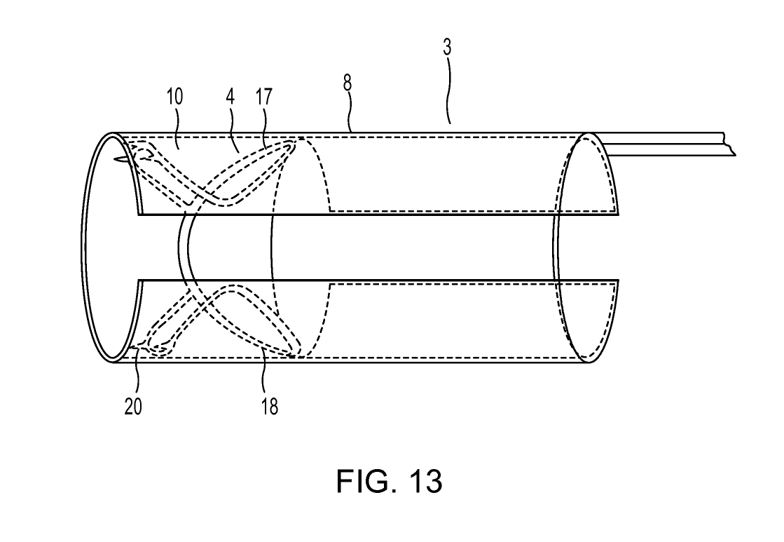

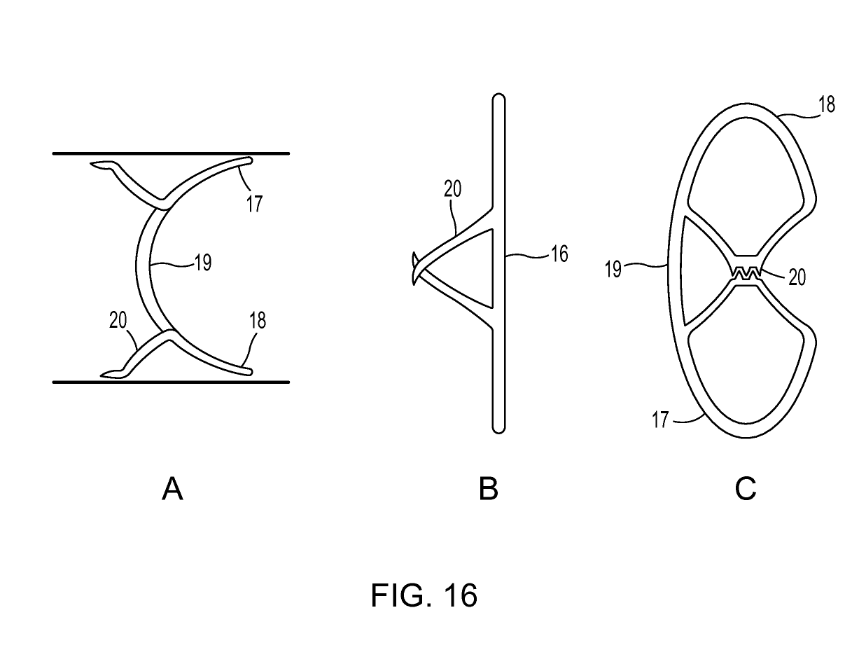

In operation to deploy the clip, a forward pressure is applied to the pusher sleeve 9 while the constraining sleeve 8 is held in place, thus pushing the clip 4 that is stored in the body 10 of the constraining sleeve 8 until the clip 4 is deployed from the delivery device 3. The constraining sleeve 8 can further include grooved channels 21 for tissue engaging members 20 of clip 4 (FIG. 15). The grooved channels 21 ensure that clip 4 will be deployed from the delivery device 3 having a specific orientation with respect to a vessel wall in a patient's body (FIG. 15). The body of the constraining sleeve can include any number of grooved channels. In certain embodiments, the number of grooved channels is equal to the number of tissue engaging members on the clip. In other embodiments, the number of grooved channels is less than the number of tissue engaging members on the clip. In other embodiments, the number of grooved channels is greater than the number of tissue engaging members on the clip. As well as ensuring the specific alignment of the clip 4 in the constraining sleeve 8, the grooved channels 21, in addition to the tapered shape of the barbs (FIG. 12), also provide the barbs 20 additional engagement thickness beyond the aperture in the vessel wall 6 that the introducer sheath goes through.

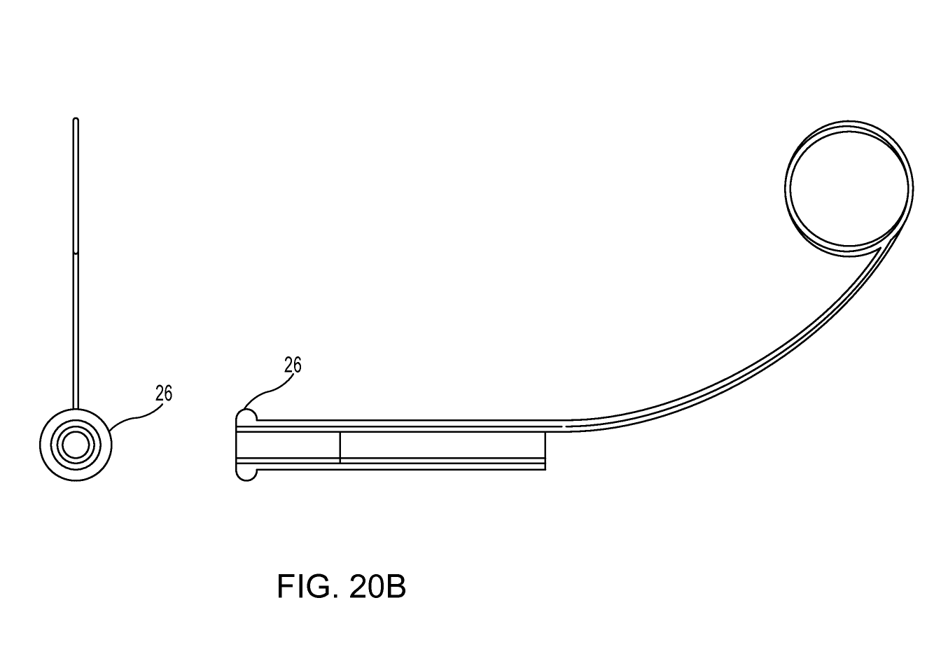

Numerous features of the delivery device prevent the clip from being deployed into an interior of the vessel. The delivery device can include a stopper that extends around at least a portion of the device, in which the stopper is positioned at a distal end of the constraining sleeve of the delivery device to prevent the pusher sleeve from advancing into the vessel. FIG. 20 panels A and B show exemplary stoppers 26 positioned at a distal end of the constraining sleeve 8, preventing the pusher sleeve 9 from advancing into the vessel 6.

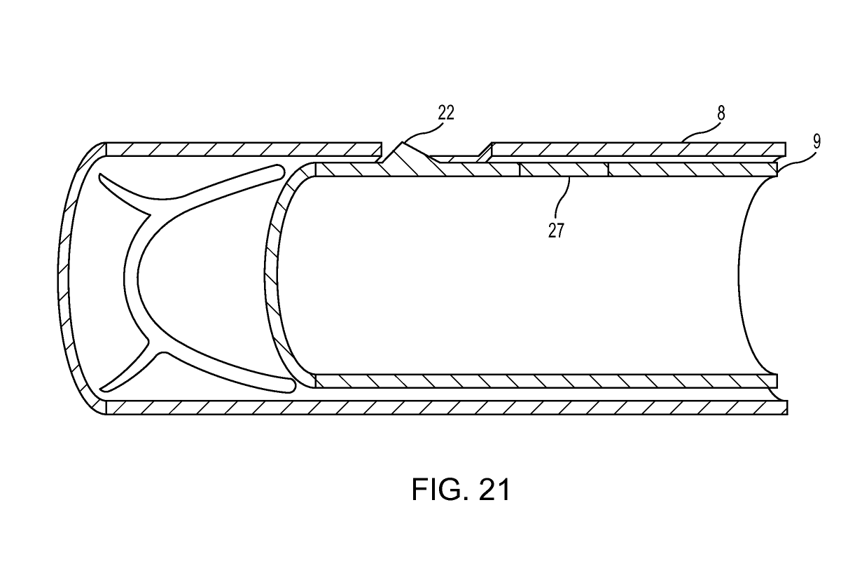

The delivery device can also include a mechanical force regulator 22 built into the delivery device. The regulator 22 generates an audible and tactile click during deployment of the clip 4 and provides feedback to the physician that deployment of the clip 4 has occurred. The mechanical force regulator 22 also ensures that the clip 4 attaches to the vessel wall 6 and is not pushed through the vessel wall 6 and into an interior space in the vessel. FIG. 21 shows a drawing of a delivery device 3 including a mechanical force regulator 22. The mechanical force regulator 22 is designed such that the pusher sleeve 9 will not advance, and thus the clip will not move inside the constraining sleeve 8, until a prescribed force is overcome.

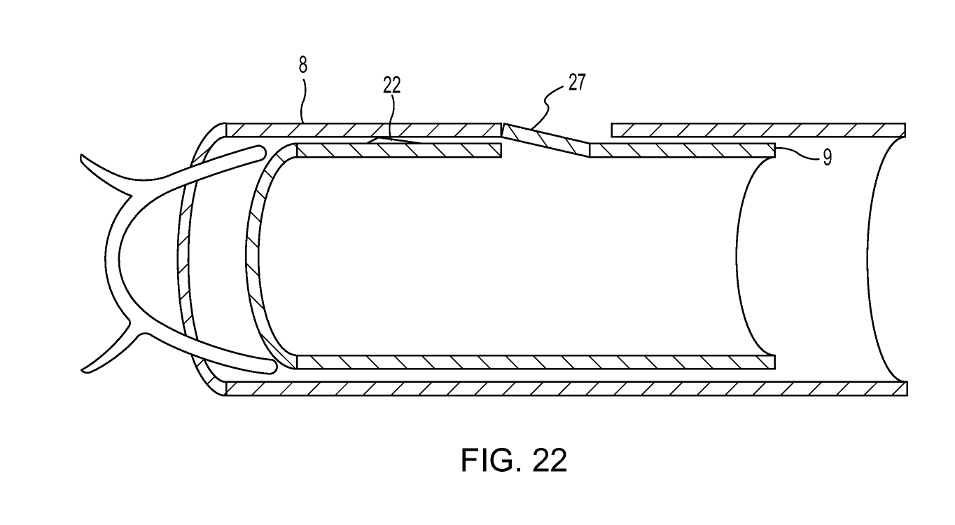

The delivery device can also include a displacement limiter 27 built into the delivery device 3. FIG. 22 shows a drawing of a delivery device 3 including a displacement limiter 27. The movement of the pusher sleeve 9 inside the constraining sleeve 8 is limited to a prescribed distance by the displacement limiter 27, thus a user cannot push the clip 4 or pusher sleeve 9 beyond a position in which the clip 4 obtains full attachment to the vessel wall 6.

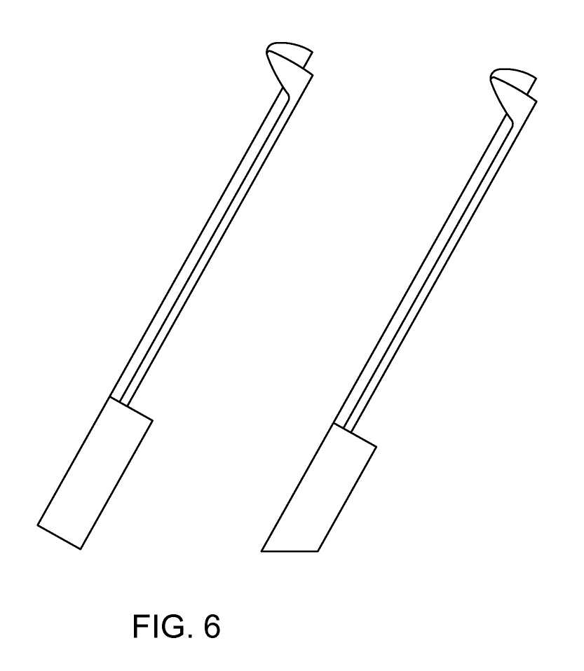

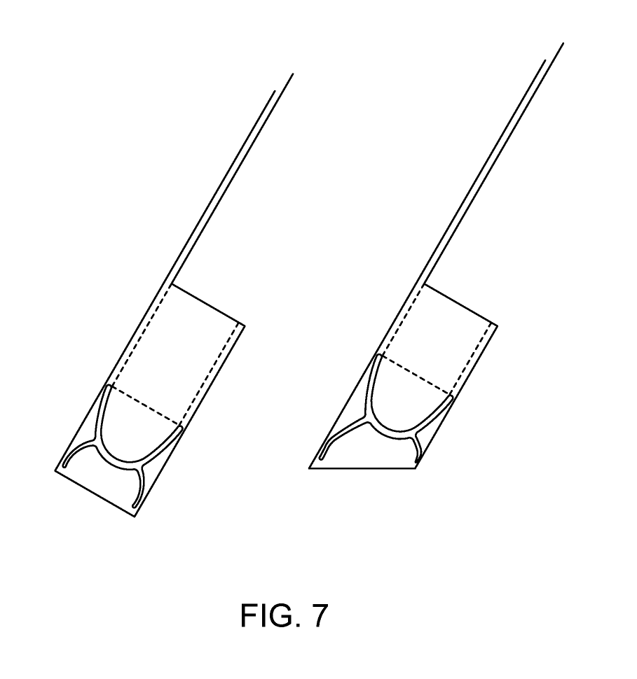

In certain embodiments, a distal end of the delivery device 3 is angled (FIG. 6 panel B compared to panel A), similar to the angle at which a surgeon may approach the vessel with the introducer sheath during such a procedure. The angled distal end of the delivery device 3 biases the loaded clip such that tissue engaging members on a first side of the clip protrude further from tissue engaging members on a second side of the clip, ensuring that upon deployment of the clip, the tissue engaging members on the first and second sides of the clip simultaneously contact an exterior of the vessel wall, as is shown in FIG. 7 panel B compared to FIG. 7 panel A. Deployment in such a manner ensures that the clip does not engage the vessel wall at an angle.

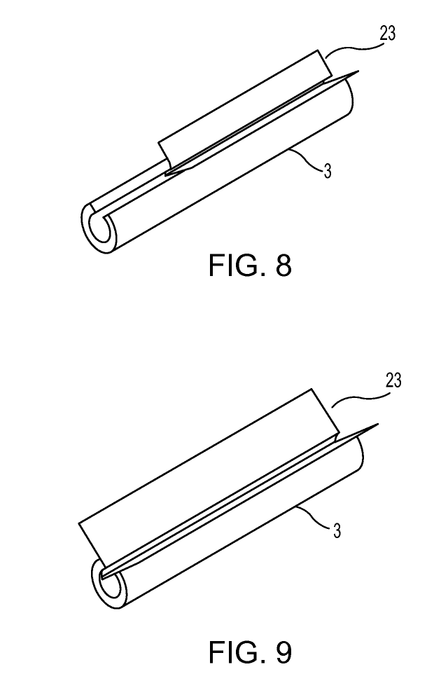

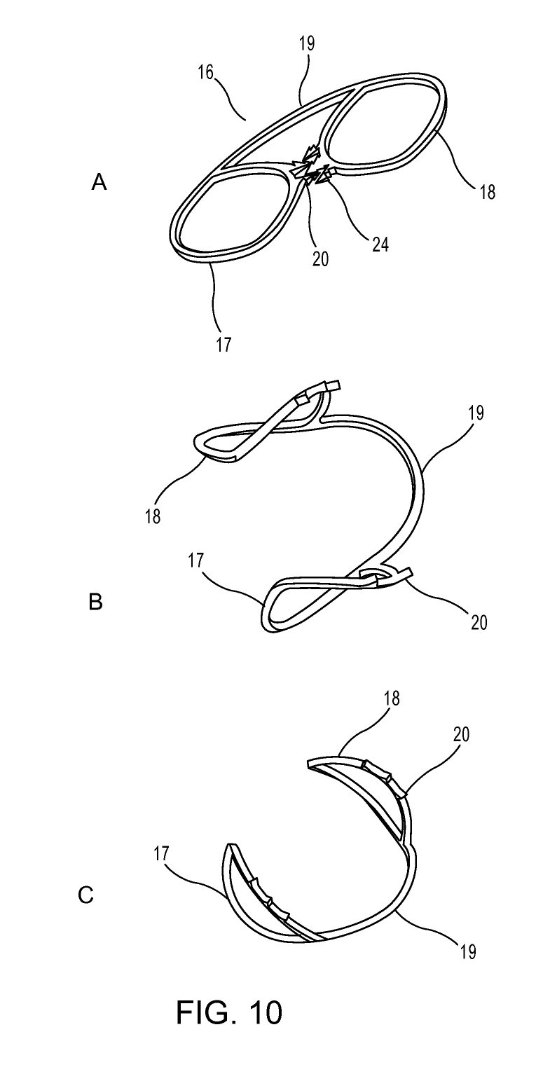

To allow attachment of the system of this invention 1 during the surgical procedure, to the introducer sheath 2, the delivery device 3 may further include a protective sheath 23 disposed along at least a portion of an interior of the clip 4 (FIGS. 8 and 9). In FIG. 8, the protective sheath 23 spans only a portion of the length of the interior of the delivery device 3. In FIG. 9, the protective sheath 23 spans a full length of the interior of the delivery device 3.

The protective sheath is capable of being peeled away from the delivery device after the delivery device has been attached to the medical device, minimizing human contact with the components that will enter a patient's body, i.e., the delivery device and the introducer sheath. In operation, the protective sheath includes a perforated seam, thus after attachment of the delivery device to the introducer sheath, the excess material is removed along the perforation prior to advancement of the delivery device into the body.

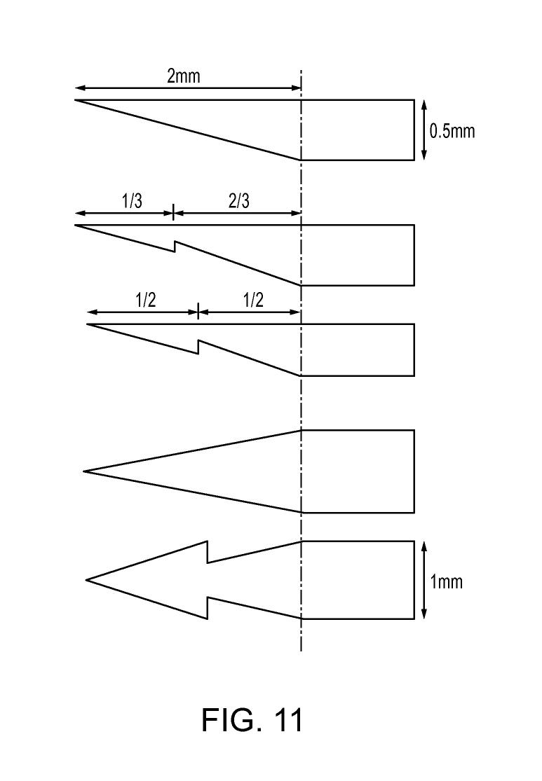

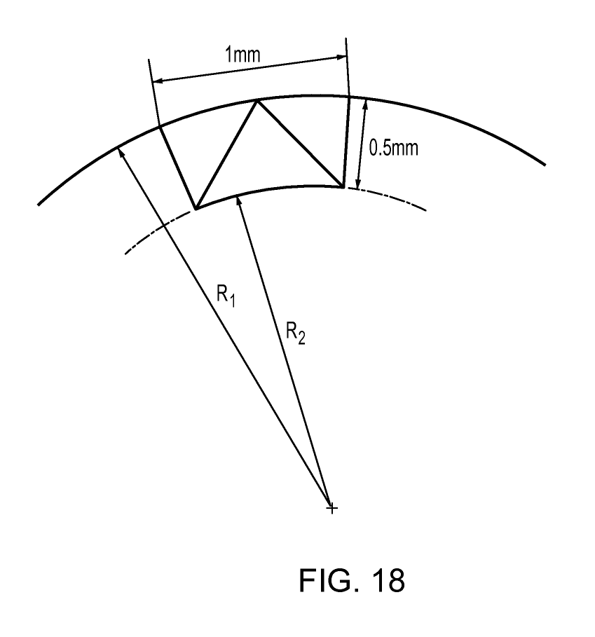

FIGS. 10 and 17 show embodiments of a clip 4 of the invention. The clip 4 can include a resilient body 16 including a first ring portion 17, a second ring portion 18, and at least one mid-region 19 joining the first and second portions (17 and 18). FIG. 10 shows an embodiment of clip 4 having a single mid region 19. FIG. 17 shows an embodiment of clip 4 having two mid-regions 19.

The first and second ring portions (17 and 18) can be any shape. For example, the first and second ring portions can be circular, square, rectangular, a polygon (regular or irregular) or a modified polygon. A modified polygon refers to shapes that include a linear portion and a non-linear portion. In certain embodiments, the first and second ring portions have the same shape. In other embodiments, the first and second ring portions have different shapes.

The clip 4 further includes at least one tissue engaging member 20 disposed about each of the first portion 17 and the second portion 18 of the body 16. In certain embodiments, the body 16 is a unitary body, as shown in FIG. 10. In other embodiments, the mid-region 19 is spring loaded. The number of tissue engaging members can be determined by one of skill in art based on the surgical intervention to be performed and the size of the instrumentation to be inserted into a patient's body. The left and right portions of the clip can include any number of tissue engaging members, for example, at least 1, at least 2, at least 3, at least 4, at least 5, at least 10, at least 20, at least 50, etc. In certain embodiments, the first and second portions (17 and 18) include a different number of tissue engaging members. For example, FIG. 10 shows clip 4 having two tissue engaging members on the left portion 17 and three tissue engaging members on the right portion 18. Alternatively, each of the first and second portions (17 and 18) can include the same number of tissue engaging members.

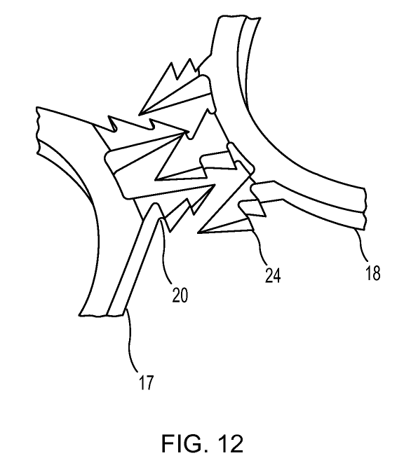

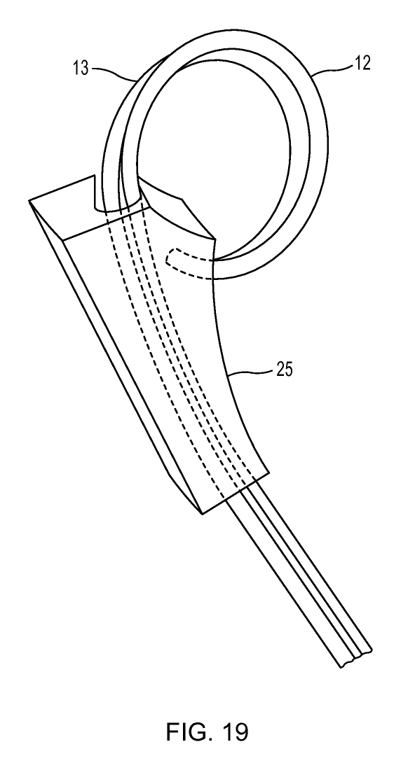

The tissue engaging members 20 can be of any shape, size or length. The shape, size and/or length of tissue engaging members 20 can be determined by one of skill in art based on the surgical intervention to be performed and the size of the instrumentation to be inserted into a patient's body. In certain embodiments, the tissue engaging members include barbs 24. FIGS. 11 and 18 provide exemplary shapes, exemplary sizes and exemplary lengths, of tissue engaging members 20. In certain embodiments, the tissue engaging members on the first portion of the body of the clip are the same shape, size, and length as the tissue engaging members on the second portion of the body of the clip. In other embodiments, the tissue engaging members on the first portion of the body of the clip are a different shape, size, and/or length as the tissue engaging members on the second portion of the body of the clip.

In certain embodiments, the clip may be fabricated from a bioresorbable or bioabsorbable material. In certain embodiments, the body 16 of the clip 4 is made from resilient materials. Exemplary resilient materials include a variety of polymers or metals, such as PLLA, PEO/PBTP, PET, PLGA, Fe, Mg, and Nitinol. The clip 4 is fabricated through methods such as molding/casting, machining, laser cutting, stereolithography, laser powder forming, fused deposition modeling, selective laser sintering, etc.

Because the body 16 of the clip 4 is made from a resilient material, the clip can have numerous configurations. In certain embodiments, the body 16 of the clip 4 has a compressed delivery configuration and an expanded deployed configuration. FIG. 10 panel A shows clip 4 in a deployed configuration, and FIG. 10 panels B and C show the clip 4 in a delivery configuration. In a deployed configuration, the tissue engaging members 20 of the first and second portions (17 and 18) interlock with each other, thereby closing the aperture in the patient's body (FIGS. 10 and 12).

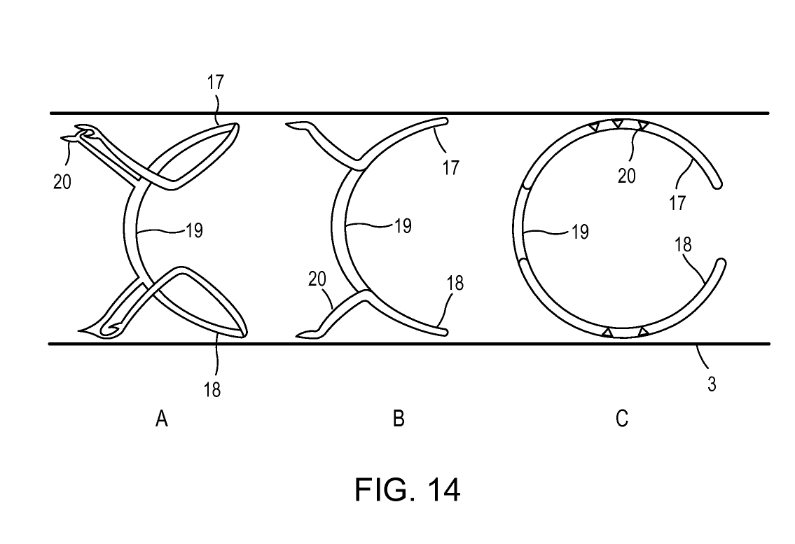

In greater detail, FIGS. 13, 14, and 16 panel A show clip 4 in a delivery configuration, i.e., the compressed configuration in which the clip is loaded into the delivery device. In the delivery configuration, the first and second portions (17 and 18) are compressed such that edges of the first and second portions (17 and 18) are directed toward a proximal end of the delivery device 3, and tissue engaging members 20 are directed away from each other and toward a distal end of the delivery device. FIG. 13 shows that in the delivery configuration, the clip 4 exerts positive pressure on the body 10 of the constraining sleeve 8 of the delivery device 3, thereby maintaining the clip 4 within the delivery device 3 until deployed by the delivery device 3. FIG. 13 further shows that in a delivery configuration, clip 4 is completely contained within delivery device 3. In this manner, delivery device 3 prevents tissue engaging members 20 of clip 4 from snagging on tissue during advancement of the delivery device 3 to the aperture in the patient's body.

Resiliency of the clip also allows the clip to be attached to different sized medical devices, such as different sized introducer sheaths. For example, introducer sheaths and/or catheters are commonly sized using the French measurement scale. The French measurement scale measures the outer diameter of cylindrical medical instruments. Due to the resiliency of the clip, the clip of the invention can be attached to a medical device of any size as measured by the French measurement system.

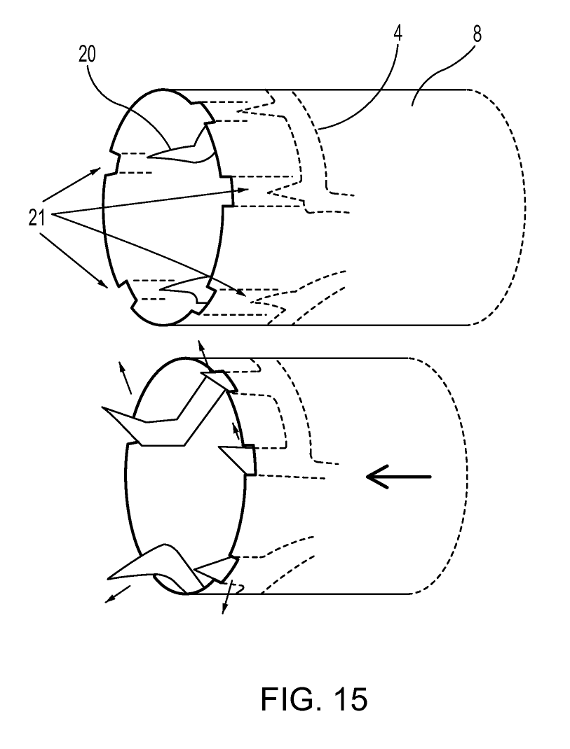

FIG. 14 panels B and C show that in the delivery configuration, clip 4 has a C-shaped cross section. It is the C-shaped cross section of the clip 4 in the delivery configuration that allows for loading of the clip 4 onto any medical device, i.e., a universal clip. Further, the C-shaped cross-section of clip 4 allows for the clip to be loaded onto a medical device after a surgical intervention has begun, without removal of the medical device from the patient's body.

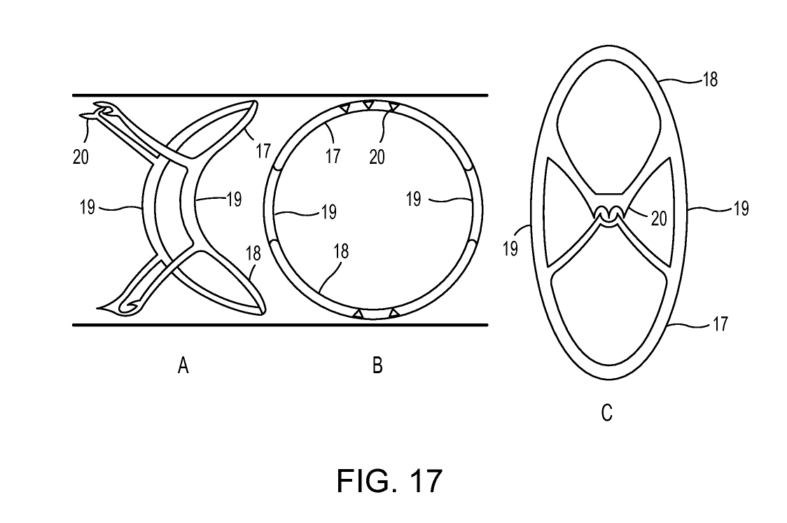

FIG. 16 panels B and C show clip 4 in a deployed configuration, i.e., the expanded configuration in which the clip engages tissue to close an aperture in a patient's body. In the deployed configuration, the body 16 of clip 4 substantially defines a plane and tissue engaging members 20 on the first and second portions (17 and 18) are directed inward toward each other (FIG. 16 panels B and C). In the deployed configuration, the tissue engaging members 20 on the first and second portions (17 and 18) lie beneath the plane defined by the body 16. Because the tissue engaging members 20 lie beneath the plane defined by the body 16 of clip 4, the clip 4 can engage an exterior vessel wall and tissue engaging members 20 will penetrate tissue surrounding the aperture in the vessel and close the aperture when the clip 4 is in a deployed configuration (FIG. 1).

Referring back to FIG. 1, methods of using a system of the invention 1 are described. In FIG. 1 panel A, introducer sheath 2 has been advanced through skin, fat, and muscle tissue into the vessel 7 through and aperture in the vessel wall 6, which is formed in accordance with well-known techniques. With the delivery device 3 situated at a proximal end of the introducer sheath 2, an interventional procedure is then performed by introducing one or more interventional devices, e.g. angioplasty balloons, stent delivery systems, atherectomy devices, etc., through the introducer sheath 2 in accordance with well-known techniques.

Upon completion of the procedure, vascular devices of the invention may be used to close the aperture in vessel wall 6 of vessel 7. The delivery device 3 is oriented on the introducer sheath 2, such that the handles 12 and 13 of delivery device 3 are in a plane that the introducer sheath 2 generates with the patient's vessel 7. The Clip 4 is oriented in the delivery system 3 such that the tissue engaging members 20 are on the medial and lateral sides of the vessel 7. The delivery device 3 is advanced over an exterior of the introducer sheath 2 through the cut-down in the skin, muscle, etc. until a distal tip of the delivery device 3 contacts the vessel 7 (FIG. 1 panel B). The physician will feel the point at which the delivery device 3 contacts the vessel 7, in that the vessel is significantly stiffer than the soft tissue between the skin and the vessel. The vessel properties vary with age of the patient, but the stiffness of the vessel in which the physician is working will have been determined while beginning the procedure and initiating access.

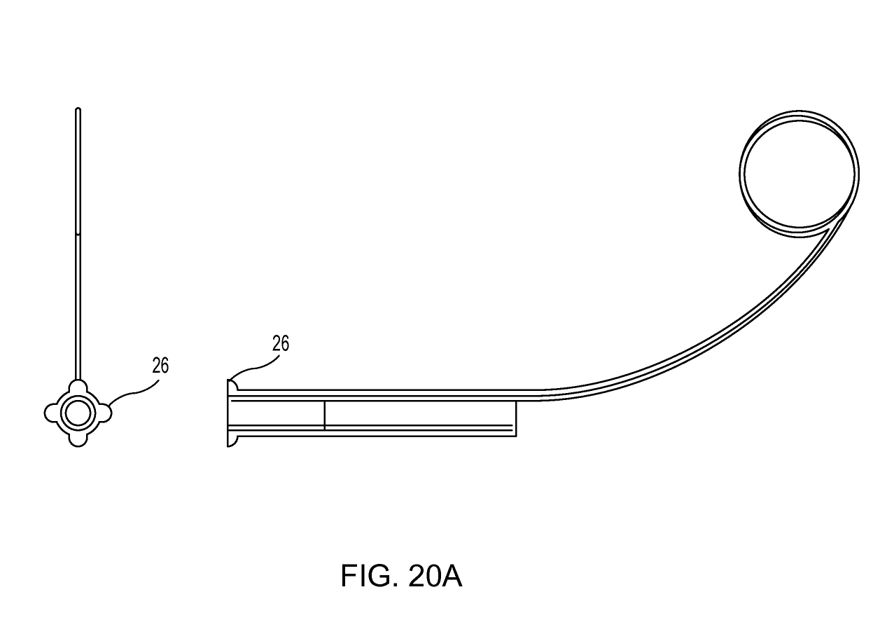

The delivery device 3 is configured such that the handles 12 and 13 are held in a position that prevents premature and/or inadvertent delivery of the clip 4, as shown in FIG. 19. The handles may be held together naturally by a user. Alternatively, a break-away tab can be used to hold the handles together. The tab is removed once the user is prepared to deploy the clip. FIG. 19 shows an embodiment in which handles 12 and 13 of delivery device 3 are held together by a tab 25, locking the handles 12 and 13 in a relative position such that deployment cannot occur until the tab 25 is removed from handles 12 and 13.

In preparation for clip deployment, the tab 25 between the handles 12 and 13, which maintains the relative position between the pusher sleeve 9 and the constraining sleeve 8, is removed while maintaining the location of the delivery device 3 at the vessel wall 6. The handle 12 of the constraining sleeve 8 is then held in one hand, along with the introducer sheath 2, while the handle 13 of the pusher sleeve 9 is held in the other hand. Again, while maintaining the position at the initial contact with the vessel wall 6 with the constraining sleeve 8 and introducer sheath 2, the pusher sleeve 9 is then advanced distally to deploy the clip 4.

The advancing of the pusher sleeve 9 requires a specified applied force before any movement occurs, at which time the pusher sleeve 9 will move forward into the fully deployed position. During this movement both an audible and tactile click will occur to signify to the physician that deployment of the clip 4 has occurred. The click will be generated by the mechanical force-regulator 22, which also ensures that the deployment is both sufficient to attach to the vessel 7 and will ensure that the clip does not penetrate through the vessel wall 6 and enter the interior space of the vessel 7.

Deployment of the clip 4 from delivery device 3 results in tissue engaging members 20 of clip 4 penetrating into the vessel wall 6 on the medial and lateral sides of the aperture. Barbs on the tissue engaging members 20 assist the tissue engaging members 20 in engaging the vessel wall 6. In certain embodiments, the tissue engaging members 20 are biased beyond a centerline of the clip 4, i.e., the tissue engaging members are biased outward, so that penetration of the tissue engaging members 20 into the vessel wall 6 begins away from the wall 6 surrounding the aperture in the vessel 7. The length of the tissue engaging members 20 are such that they are approximately the thickness of the vessel wall 6, so in some embodiments the tissue engaging members 20 will slightly protrude into the interior of the vessel. A base of each tissue engaging member 20 is large enough to limit movement so that the clip does not penetrate through the vessel wall 6.

At this stage of deployment, the clip 4 is still in the delivery configuration. As the introducer sheath 2 is withdrawn from the vessel 7, the clip 4 expands to its deployed configuration in which the body 16 of the clip 4 is flat and in a plane and tissue engaging members 20, in some embodiments may lie in the same plane while in other embodiments may lie below the plane, while they engage the exterior vessel wall 6 (FIG. 1 panel C and FIG. 16 panel B). The transition of the clip 4 from the delivery configuration to the deployed configuration pulls vessel wall tissue on each side of the aperture together to close the aperture in the vessel 7. The clip 4 in the deployed configuration has the tissue engaging members 20 directed toward each other and interlocking with each other.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including the references to the scientific and patent literature cited herein.

What is claimed is:

1. A method for closing an aperture in a vessel in a patient's body, the method comprising:
advancing a first medical device through an aperture in a vessel;
advancing a delivery device distally along an exterior surface of the medical device to contact an exterior vessel wall; and
deploying a clip that engages tissues portions adjacent to the aperture in the vessel and closes the aperture in the vessel upon withdrawal of the first medical device from the aperture.

2. The method according to claim 1, wherein prior to advancing the first medical device through the aperture in the vessel, the method further comprises attaching the delivery device to an exterior surface of the first medical device.

3. The method according to claim 1, wherein after advancing the first medical device through the aperture in the vessel, the method further comprises attaching the delivery device to an exterior surface of the first medical device.

4. The method according to claim 1, wherein the clip is resilient.

5. The method according to claim 4, wherein the clip is expandable from a delivery configuration in which the clip is loaded within the delivery device to a deployed configuration in which opposite ends of the clip are directed inward towards each other.

6. The method according to claim 5, wherein withdrawal of the medical device results in tissue engaging portions of the clip to move toward each other and interlocking with each other, closing the aperture in the vessel.

7. The method according to claim, 1, wherein the clip is bioabsorbable or bioresorbable.

8. The method according to claim 1, wherein the first medical device is an introducer sheath.

9. The method according to claim 8, further comprising introducing at least a second medical device through the introducer sheath into the vessel.

10. The method according to claim 9, wherein the second medical device is selected from the group consisting of an angioplasty balloon, an atherectomy device, a IVE filter, a dialysis filter, and a stent delivery device.

11. The method according to claim 9, further comprising performing a surgical intervention within the patient's body using the second medical device introduced through the introducer sheath into the vessel.

12. The method according to claim 1, further comprising delivering a tissue sealant into the aperture.

13. The method according to claim 1, wherein the delivery device comprises a pusher sleeve and a constraining sleeve, wherein the pusher sleeve is slidably disposed within the constraining sleeve.

14. The method according to claim 13, wherein deploying the clip comprises pushing the pusher sleeve to advance the clip from the delivery device.

15. A method for closing an aperture in a vessel in a patient's body, the method comprising:
advancing a first medical device through an aperture in a vessel;
advancing a delivery device distally along an exterior surface of the medical device to contact an exterior vessel wall; and
deploying a biodegradable clip that engages tissues portions adjacent to the aperture in the vessel and closes the aperture in the vessel upon withdrawal of the first medical device from the aperture.

16. A method for closing an aperture in a vessel in a patient's body, the method comprising:
advancing a delivery device distally along an exterior surface of a medical device to contact an exterior vessel wall; and
deploying a clip that engages tissues portions adjacent to the aperture in the vessel and closes the aperture in the vessel upon withdrawal of the medical device from the aperture.

17. A method for closing an aperture in a vessel in a patient's body, the method comprising:
advancing a medical device through an aperture in a vessel;
attaching a delivery device to an exterior surface of the medical device after the medical device has been advanced through the aperture in the vessel;
advancing the delivery device distally along the exterior surface of the medical device to contact an exterior vessel wall; and
deploying a clip from the delivery device that engages tissues portions adjacent to the aperture in the vessel and closes the aperture in the vessel upon withdrawal of the medical device from the aperture.
